(19) United States
(12) Patent Application Publication
(10) Pub. No.: US 2016/0205798 A1
(43) Pub. Date: Jul. 14, 2016

(54) WATERTIGHT CASING WITH INTEGRATED ELECTRICAL CONTACTS

(71) Applicant: LifeScan Scotland Limited, Inverness (GB)

(72) Inventors: GUINAN Eamon, Inverness (GB); NELSON Jonathan, Inverness (GB); ELDER David, Inverness (GB); FAULKNER Allan, Avoch (GB); HAMER Malcolm, Inverness (GB); MAGENNIS Ryan, Beauly (GB)

(21) Appl. No.: 14/911,498

(22) PCT Filed: Aug. 11, 2014
(86) PCT No.: PCT/EP2014/067185

§ 371 Date: Feb. 11, 2016

(30) Foreign Application Priority Data
Aug. 12, 2013 (GB) 1314400.1

(51) Int. Cl. H05K 5/06; G01N 27/327; G01N 33/49; G01N 27/416; H05K 5/00; G01N 33/487

(52) U.S. Cl. CPC H05K 5/069; H05K 5/0086; G01N 33/48785; G01N 33/49; G01N 27/4166; G01N 27/3275

(57) ABSTRACT

A watertight casing for an electronic device includes an electrically-insulating plastic case (EIPC) and at least one electrically-conductive thermoplastic elastomer (ECTPE) contact. The EIPC includes inwardly and outwardly facing surfaces. The ECTPE contact that includes a proximal contact portion with a proximal contact surface, a distal contact portion with a distal contact surface, and a channel portion connecting the proximal contact portion and the distal contact portion. The ECTPE contact(s) is integrated with the EIPC such that the ECTPE contact(s) extends through the EIPC from the outwardly facing surface to the inwardly facing surface and such that the proximal contact surface is disposed in proximity to the inwardly facing surface and the distal contact surface is disposed in proximity to the outwardly facing surface. The ECTPE contact(s) is adhered to the EIPC such that a watertight seal between the outwardly facing surface and the inwardly facing surface is present therebetween.

### FIELD OF THE INVENTION

The present invention relates, in general, to electronic devices and, in particular, to electronic device casings that include electrical contacts and associated methods.

### BACKGROUND OF THE INVENTION

The determination (e.g., detection and/or concentration measurement) of an analyte in, or a characteristic of, a fluid sample is of particular interest in the medical field. For example, it can be desirable to determine glucose, ketone bodies, cholesterol, lipoproteins, triglycerides, acetaminophen, hematocrit and/or HbA1c concentrations in a sample of a bodily fluid such as urine, blood, plasma or interstitial fluid. Such determinations can be achieved using a hand-held test meter and associated analytical test strips that employ, for example, visual, photometric or electrochemical determination techniques. Such hand-held test meters, as well as a variety of other electronic devices, are typically configured to hold and connect with batteries (e.g., coin cell batteries) that are employed as the device's power supply and/or to electrically connect with a variety of associated items such as, for example, analytical test strips.

### SUMMARY OF THE INVENTION

In a first aspect of the invention, there is provided a watertight casing for an electronic device, the watertight casing comprising: an electrically-insulating plastic case with: an inwardly facing surface; and an outwardly facing surface; at least one electrically-conductive thermoplastic elastomer (ECTPE) contact that includes: a proximal contact portion with a proximal contact surface; a distal contact portion with a distal contact surface, and a channel portion connecting the proximal contact portion and the distal contact portion, wherein the at least one ECTPE contact is integrated with the electrically-insulating plastic case such that the ECTPE contact extends through the electrically-insulating plastic case from the outwardly facing surface to the inwardly facing surface; and wherein the proximal contact surface is disposed in proximity to the inwardly facing surface and the distal contact surface is disposed in proximity to the outwardly facing surface; and wherein the at least one ECTPE contact is adhered to the electrically-insulating plastic case such that a watertight seal between the outwardly facing surface and the inwardly facing surface is present therebetween.

The electrically-insulating plastic case may be configured as at least a portion of a hand-held test meter for the determination of an analyte in a bodily fluid sample.

The electrically-insulating plastic case may be configured as a strip port connector of the hand-held test meter.

The at least one ECTPE contact may be configured to contact an electrochemical-based analytical test strip inserted into the strip port connector and a printed circuit board within the hand-held test meter.

The electrically-insulating plastic case may be configured as a battery compartment of the hand-held test meter.

The plurality of ECTPE contacts may be configured to contact a battery inserted into the battery compartment and a printed circuit board within the hand-held test meter.

The at least one ECTPE contact may be formed of an ECTPE material that includes a non-conductive polymer randomly doped with a conductive particle.

The conductive particle may be a carbon black particle.

The at least one ECTPE material may be elastically deformable.

The proximal contact portion may have a raised profile with respect to the inwardly facing surface and the distal contact portion may have a raised profile with respect to the outwardly facing surface.

At least the proximal contact portion and the distal contact portion may be elastically deformable.

In a second aspect of the invention there is provided a method for handling an electronic device, the method comprising: handling an electronic device that includes a watertight casing with: an electrically-insulating plastic case; and at least one electrically-conductive thermoplastic elastomer (ECTPE) contact, such that an outwardly facing surface of the watertight casing and a distal contact portion of the ECTPE contact in proximity to the outwardly facing surface are subjected to moisture, and preventing the moisture from reaching an inwardly facing surface of the electrically-insulating plastic case and a proximal surface of the ECTPE contact in proximity thereto in a passive manner due to the ECTPE contact being integrated with the electrically-insulating plastic case such that the ECTPE contact extends through the electrically-insulating plastic case from the outwardly facing surface to the inwardly facing surface with the ECTPE contact being adhered to the electrically-insulating plastic case such that a watertight seal between the outwardly facing surface and the inwardly facing surface is present therebetween.

The handling may also be such that the outwardly facing surface of the watertight casing and a distal contact portion of the ECTPE contact in proximity to the outwardly facing surface are subjected to dust, and the preventing may also be such that the dust is prevented from reaching an inwardly facing surface of the watertight casing and a proximal surface of the ECTPE contact in proximity thereto in a passive manner due to the ECTPE contact being integrated with the electrically-insulating plastic case.

The electrically-insulating plastic case may be configured as at least a portion of a hand-held test meter for the determination of an analyte in a bodily fluid sample.

The electrically-insulating plastic case may be configured as a strip port connector of the hand-held test meter.

The at least one ECTPE contact may be configured to contact an electrochemical-based analytical test strip inserted into the strip port connector and a printed circuit board within the hand-held test meter.

The electrically-insulating plastic case may be configured as a battery compartment of the hand-held test meter.

The plurality of ECTPE contacts may be configured to contact a battery inserted into the battery compartment and a printed circuit board within the hand-held test meter.

The at least one ECTPE contact may be formed of an ECTPE material that includes a non-conductive polymer randomly doped with a conductive particle.

The conductive particle may be a carbon black particle.

The at least one ECTPE material may be elastically deformable.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified cross-sectional view of a portion of a watertight casing with integrated electrical contacts according to an embodiment of the present invention operably contacting a printed circuit board and with an electrochemical-based analytical test strip inserted therein;

FIG. 2 is a simplified cross-sectional view of a watertight casing with integrated electrical contacts according to another embodiment of the present invention operably contacting a printed circuit board, with an electrochemical-based analytical test strip inserted therein and joined to an associated plastic case (e.g., a housing of a hand-held test meter);

FIG. 3 is a simplified perspective view of a watertight casing with integrated electrical contacts according to an additional embodiment of the present invention;

FIG. 4 is another simplified perspective view of the watertight casing of FIG. 3;

FIG. 5 is yet another simplified perspective view of the watertight casing of FIG. 3;

FIG. 6 is yet another simplified front view of the watertight casing of FIG. 3;

FIG. 7 is yet another simplified cross-sectional view of a portion of the watertight casing of FIG. 3 joined to an associated plastic case (e.g., a housing of a hand-held test meter);

FIG. 8 is a simplified perspective view of the watertight casing of FIG. 3 with an electrochemical-based analytical test strip inserted therein;

FIG. 9 is a simplified front view of the watertight casing of FIG. 3 with an electrochemical-based analytical test strip inserted therein;

FIG. 10 is a perspective view of a watertight casing with integrated electrical contacts according to a further embodiment of the present invention along with an associated coin cell battery and lid;

FIG. 11 is another perspective view of the watertight casing with integrated electrical contacts of FIG. 10 along with the associated coin cell battery and lid;

FIG. 12 is a perspective view of a watertight casing with integrated electrical contacts according to a further embodiment of the present invention;

FIG. 13 is a cross-sectional perspective view of the watertight casing with integrated electrical contacts of FIG. 10;

FIG. 14 is another simplified perspective view of the watertight casing with integrated electrical contacts of FIG. 10;

FIG. 15 is a cross-sectional perspective view of the watertight casing with integrated electrical contacts of FIG. 10 operably contacting a printed circuit board;

FIG. 16 is a cross-sectional perspective view of a portion of the watertight casing with integrated electrical contacts of FIG. 10 operably contacting a printed circuit board;

FIG. 17 is a cross-sectional view of a portion of the watertight casing with integrated electrical contacts of FIG. 10 operably contacting a printed circuit board;

FIG. 18 is a simplified perspective view of the watertight casing with integrated electrical contacts of FIG. 10 operably contacting a printed circuit board and with a battery being inserted therein;

FIG. 19 is a simplified perspective view of the watertight casing with integrated electrical contacts of FIG. 10 operably contacting a printed circuit board and with a battery fully inserted therein;

FIG. 20 is a simplified cross-sectional perspective view of the watertight casing with integrated electrical contacts of FIG. 10 operably contacting a printed circuit board and with a battery fully inserted therein;

FIG. 21 is a simplified cross-sectional view of a portion of the watertight casing with integrated electrical contacts of FIG. 10 operably contacting a printed circuit board and with a battery fully inserted therein; and

FIG. 22 is a flow diagram depicting stages in a method for employing an electronic device according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, watertight casings (e.g., a battery compartment casing or a strip port connector casing) for an electronic device (for example, a hand-held test meter) according to embodiments of the present invention include an electrically-insulating plastic case and at least one electrically-conductive thermoplastic elastomer (ECTPE) contact. The electrically-insulating plastic case includes inwardly and outwardly facing surfaces. The ECTPE contact that includes a proximal contact portion with a proximal contact surface, a distal contact portion with a distal contact surface, and a channel portion connecting the proximal contact portion and the distal contact portion. In addition, the ECTPE contact(s) is integrated with the electrically-insulating plastic case such that the ECTPE contact(s) extends through the electrically-insulating plastic case from the outwardly facing surface to the inwardly facing surface and such that the proximal contact surface is disposed in proximity to the inwardly facing surface and the distal contact surface is disposed in proximity to the outwardly facing surface. Moreover, the ECTPE contact(s) is adhered to the electrically-insulating plastic case such that a watertight seal between the outwardly facing surface and the inwardly facing surface is present therebetween.

As used herein, the terms “case” and “casing” refer to an outer covering or housing and the term “watertight” indicates that the described item is of such tight construction or fit as to be impermeable to water, other liquids and dust. The term “adhered” refers to any of, or a combination of, chemical bonding, inter-diffusion and mechanical interlocking. Adherence between the ECTPE contacts and the electrically-insulating plastic case can be achieved by, for example, forming the ECTPE contacts and electrically-insulating plastic case using co-injection molding (also referred to as two shot molding).

Watertight casings according to embodiments of according to embodiments of the present invention are beneficial in that, for example, they prevent water and dust from passing through the casing, along any interface between the electrical contacts and the electrically-insulating case, and entering the interior of the casing where the water and/or dust is at risk of harming electronics. Moreover, the watertight casings according to embodiments of the present invention are relatively low-cost, simple to manufacture and to easily connected to other components of the electronic device (such as a printed circuit board (PCB)) using solder-less techniques. Moreover, the outwardly facing surface of watertight casings according to the embodiments of the present invention can be beneficially cleaned with water or other suitable liquids without the risk of such water or other liquids inadvertently leaking to the inwardly facing surface and thereafter contaminating electronics or other electronic device components on the inwardly facing side of the watertight casing (i.e., on the inside of the electronic device).

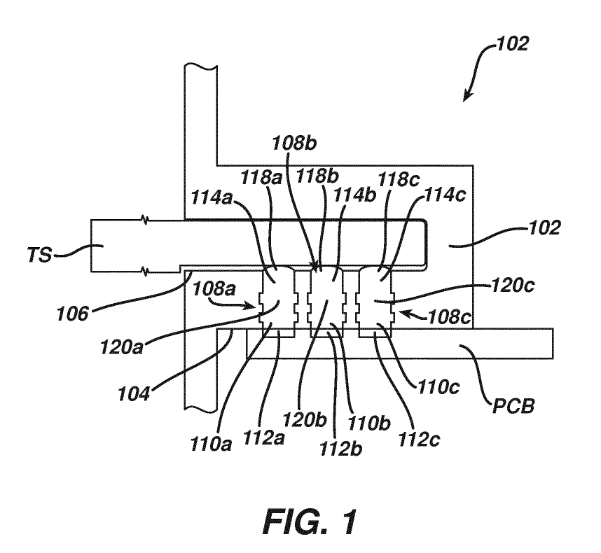

FIG. 1 is a simplified cross-sectional view of a portion of a watertight casing with integrated electrical contacts 100 (also referred to simply as a “watertight casing”) for use in an electronic device (i.e., a hand-held test meter) according to an embodiment of the present invention. In FIG. 1, watertight casing 100 is operably contacting a printed circuit board (PCB) and has an electrochemical-based analytical test strip (TS) inserted therein.

Referring to FIG. 1, watertight casing 100 includes an electrically-insulating plastic case 102 with an inwardly facing surface 104 and an outwardly facing surface 106. Watertight casing 100 also includes three electrically-conductive thermoplastic elastomer (ECTPE) contacts 108a, 108b and 108c.

Each of these ECTPE contacts includes a proximal contact portion (110a, 110b and 110c, respectively) with a proximal contact surface (112a, 112b, and 112c, respectively), a distal contact portion (114a, 114b, and 114c, respectively) with a distal contact surface (118a, 118b, and 118c, respectively), and a channel portion (120a, 122b, and 122c, respectively) connecting the proximal contact portion and the distal contact portion of each ECTPE contact.

As is evident from FIG. 1, each of the ECTPE contacts (108a, 108b, and 108c) is integrated with the electrically-insulating plastic case 102 such that the ECTPE contacts extend through electrically-insulating plastic case 102 from the outwardly facing surface 106 to the inwardly facing surface 104. In addition, each of the proximal contact surfaces (112a, 112b, and 112c) is disposed in proximity to the inwardly facing surface 104 and the distal contact surfaces (118a, 118b and 118c) are disposed in proximity to the outwardly facing surface 106. The distal contact surfaces are disposed such that they make operable electrical contact with the electrochemical-based analytical test strip that is inserted into watertight casing 100 vie, for example, mechanical forces that elastically deform the distal contact surfaces against corresponding contact pads (not shown) of the electrochemical-based analytical test strip.

Each of the ECTPE contacts (108a, 108b and 108c) is adhered to the electrically-insulating plastic case such that a watertight seal between the outwardly facing surface 106 and the inwardly facing surface 104 is present therebetween. Such a watertight seal is present at the interface of the ECTPE contacts and the electrically-insulating case and is a result of forming the watertight casing using, for example, a co-injection technique.

Operable electrical contact between the ECTPE contacts (108a, 108b and 108c) and solder pads of the PCB is securely established by elastic deformation of the ECTPE being pressed against solder pads of the SPC. The ETCPE contacts are pressed against the PCB by the predetermined configuration of the electrically-insulating case. Watertight case 100 can be mounted onto the PCB using any suitable technique including, for example, heat staking, surface mount technology or some other mechanically sound method of fixation.

ECTPE contacts 108a, 108b and 108c can be formed of any suitable ECTPE material. Suitable ECTPE materials include a durable thermoplastic rubber doped with electrically conductive micro-particles or electrically-conductive nano-particles. The effect of randomly doping a non-conductive polymer with an electrically conductive particle, such as carbon black, is such that once the volume fraction of the electrically conductive particle reaches a critical value, Φ_{c}, the resistivity of the composite material falls rapidly.

The equation below describes the electrical conductivity of a random dispersion polymer composite, ρ, for different values of:
Φ=volume fraction of electrically conductive particle filler material
ρ=conductivity of electrically conductive particle filler material
β=geometry factor (empirically derived for a particular electrically conductive particle filler material, shape, and size distribution).

ρ=ρ₀(Φ−Φ_{c})^{-β}

This equation illustrates that the conductivity of the ECTPE material can be predetermined based on the electrically conductive particle geometry (size and shape), particle conductivity and particle volume fraction. Suitable electrically conductive particles for inclusion in an ECTPE material include, but are not limited to, carbon particles, carbon nanotubes, silver coated microspheres, nickel coated graphite, and metallic particles.

ECTPE materials employed in embodiments of the present invention can be any suitable ECTPE material including, as a non-limiting example, an ECTPE material commercially available from Premix as “Preseal TPE.” To create a watertight seal between the ECTPE contacts and the electrically-insulating plastic case, the ECTPE should have characteristics that provide for watertight seal to be formed upon such co-injection. Once apprised of the present disclosure, suitable adherence can be determined by one skilled in the art via routine experimentation.

Electrically-insulating plastic casing 102 can be formed of any suitable plastic material including, for example, polypropylene, polystyrene and polycarbonate, poly(methyl methacrylate) (PMMA), polyoxymethylene (POM), acrylonitrile butadiene styrene (ABS), a glass-reinforced liquid crystal polymer (LCP) and combinations thereof. The plastic material of the electrically-insulating casing is selected such that it is compatible with, for example has operable adhesion with, the ECTPE material.

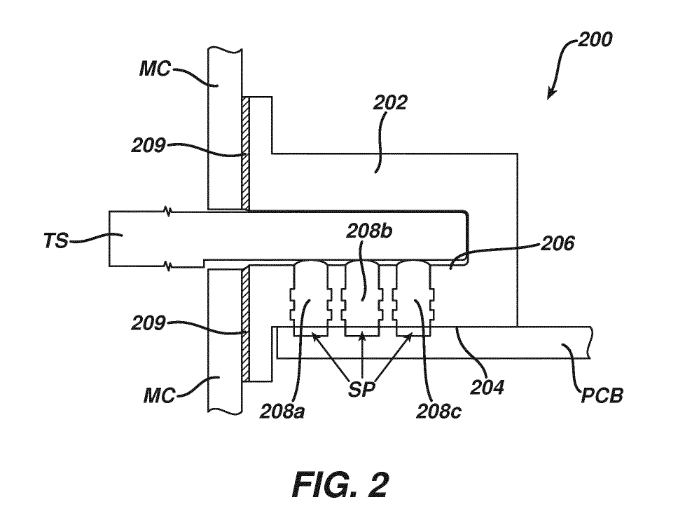

FIG. 2 is a simplified cross-sectional view of a watertight casing with integrated electrical contacts 200 (also referred to simply as a “watertight casing”) according to another embodiment of the present invention operably contacting solder pads (SP) a printed circuit board PCB, with an electrochemical-based analytical test strip TS inserted therein and joined to an associated plastic case (MC) of a hand-held test meter. The PCB includes a plurality of solder pads (each labeled SP) configured for operable contact with the integrated electrical contacts (i.e., the ECTPE contacts described below).

Referring to FIG. 2, watertight casing 200 includes an electrically-insulating plastic case 202 with an inwardly facing surface 204 and an outwardly facing surface 206. Watertight casing 200 also includes three electrically-conductive thermoplastic elastomer (ECTPE) contacts 208a, 208b and 208c and a gasket 209.

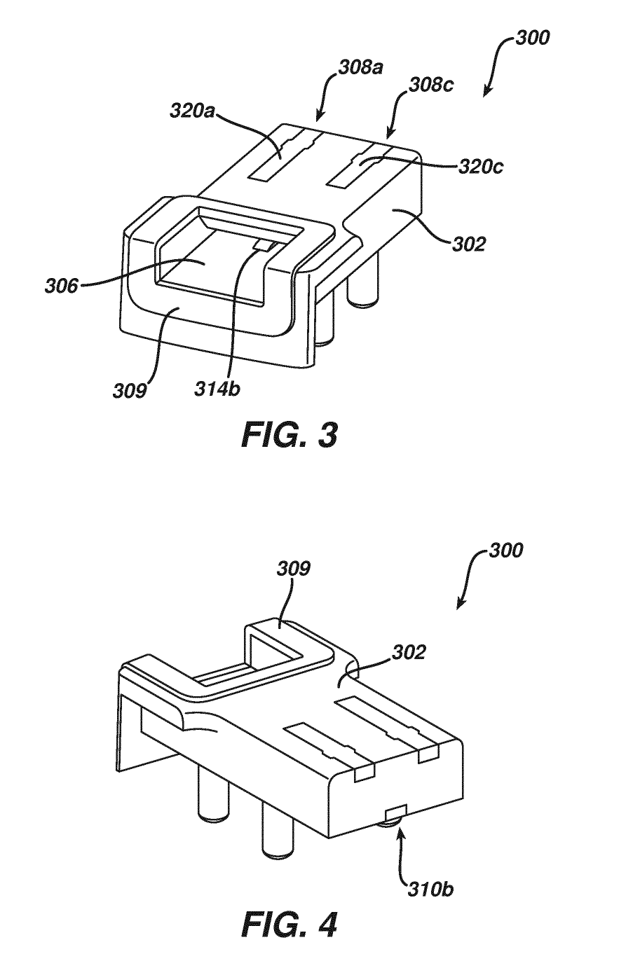

The ECTPE contacts of watertight casing 200 are identical to those of watertight casing 100. However, electrically-insulating plastic case 202 is configured, along with gasket 209, to operably attach to, and be detached from, associated plastic case MC. Therefore, watertight casing 200 can be considered a strip port connector (SPC) sub-assembly or SPC component of a hand-held test meter. Such a test meter would employ the associated plastic case MC and PCB of FIG. 3 as additional sub-assemblies or components of a complete hand-held test meter (not depicted in the figures).

Gasket 209 can be formed of any suitable material including suitable natural rubbers and suitable thermo-elastic materials. Moreover, gasket 209 is configured to provide a watertight seal between electrically-insulating plastic case 202 and associated hand-held meter case MC (also referred to as a housing), thereby providing a fully watertight housing for a hand-held test meter once watertight casing 200 and associated meter case MC are connected.

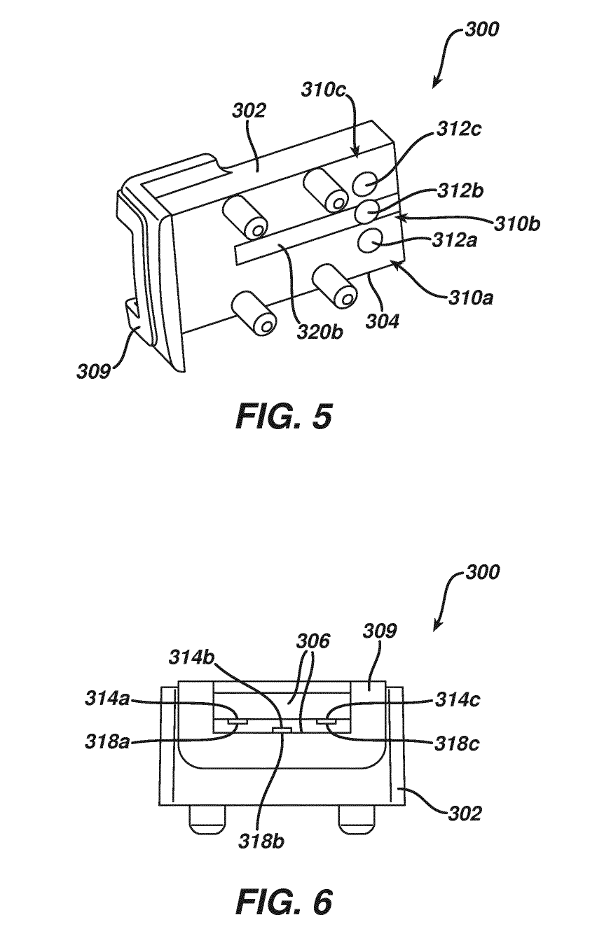

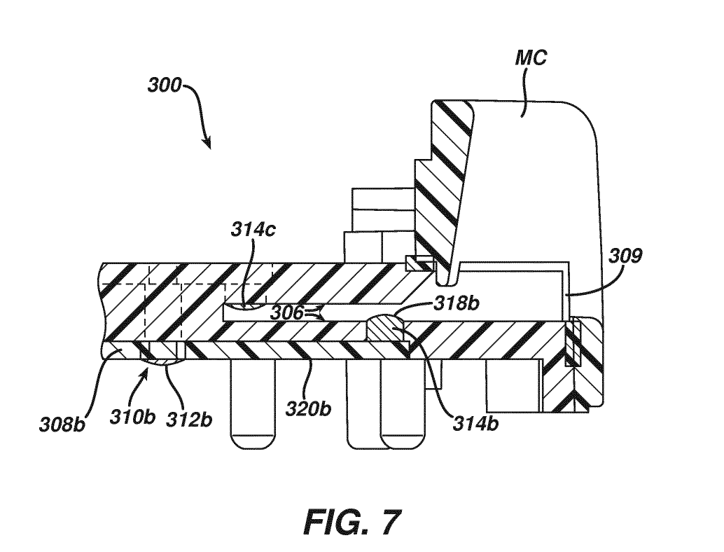

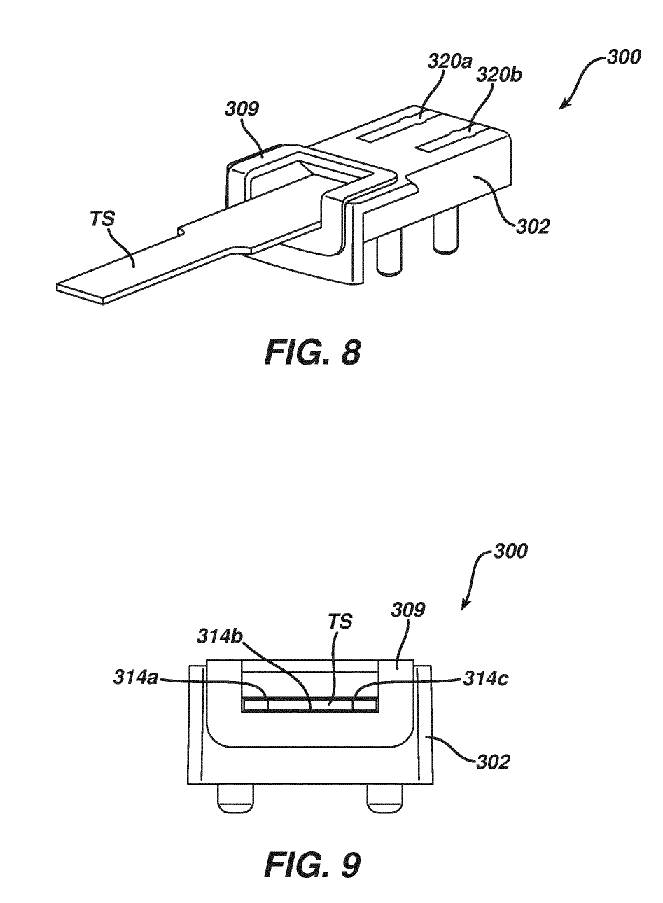

FIG. 3 is a simplified perspective view of a watertight casing with integrated electrical contacts 300 (also referred to simply as a “watertight casing”) according to an additional embodiment of the present invention. FIG. 4 is another simplified perspective view of watertight casing 300. FIG. 5 is yet another simplified perspective view of watertight casing 300. FIG. 6 is yet another simplified front view of watertight casing 300. FIG. 7 is yet another simplified cross-sectional view of a portion of watertight casing 300 sealed against associated meter casing MC. FIG. 8 is a simplified perspective view of watertight casing 300 with an electrochemical-based analytical test strip TS operably inserted therein. FIG. 9 is a simplified front view of watertight casing 300 with an electrochemical-based analytical test strip TS operably inserted therein.

Referring to FIGS. 3 through 9, watertight casing 300 includes an electrically-insulating plastic case 302 with an inwardly facing surface 304 and an outwardly facing surface 306. Watertight casing 300 also includes three electrically-conductive thermoplastic elastomer (ECTPE) contacts 308a, 308b and 308c. Watertight casing 300 also includes a gasket 309.

Each of these ECTPE contacts includes a proximal contact portion (310a, 310b and 310c, respectively) with a proximal contact surface (312a, 312b, and 312c, respectively), a distal contact portion (314a, 314b, and 314c, respectively) with a distal contact surface (318a, 318b, and 318c, respectively), and a channel portion (320a, 320b, and 320c, respectively) connecting the proximal contact portion and the distal contact portion of each ECTPE contact.

As is evident from FIGS. 3-9, each of the ECTPE contacts (308a, 308b, and 308c) is integrated with the electrically-insulating plastic case 302 such that the ECTPE contacts extend through electrically-insulating plastic case 302 from the outwardly facing surface 306 to the inwardly facing surface 304 (see FIG. 7 in particular). In addition, each of the proximal contact surfaces (312a, 312b, and 312c) is disposed in proximity to the inwardly facing surface 304 and the distal contact surfaces (318a, 318b and 318c) are disposed in proximity to the outwardly facing surface 306. The distal contact surfaces are disposed such that they make operable electrical contact with the electrochemical-based analytical test strip TS that is inserted into watertight casing 300.

Each of the ECTPE contacts (308a, 308b and 308c) is adhered to the electrically-insulating plastic case such that a watertight seal between the outwardly facing surface 306 and the inwardly facing surface 304 is present therebetween.

Referring to FIGS. 6 and 9 in particular, the elastic nature and configuration of the ECTPE contacts results in the ECTPE contacts elastically deforming as the test strip is inserted. The ECTPE contacts are of a suitable dimension so as to apply an appropriate contact force to the test strip surface. A typical but non-limiting insertion force for the test strip is 4N with such force being distributed essentially evenly across the ECTPE contacts.

Once apprised of the present disclosure, one skilled in the art will recognize that watertight casing 100, watertight casing 200 and watertight casing 300 are configured to operate as a Strip Port Connector (SPC) for an associated hand-held test meter for the determination of an analyte (such as glucose) in a bodily fluid sample (e.g., a whole blood sample) using a test strip (for example, an electrochemical-based analytical test strip). FIGS. 2 and 7 show watertight casing sealed against the meter housing (MC) of such a hand-held test meter.

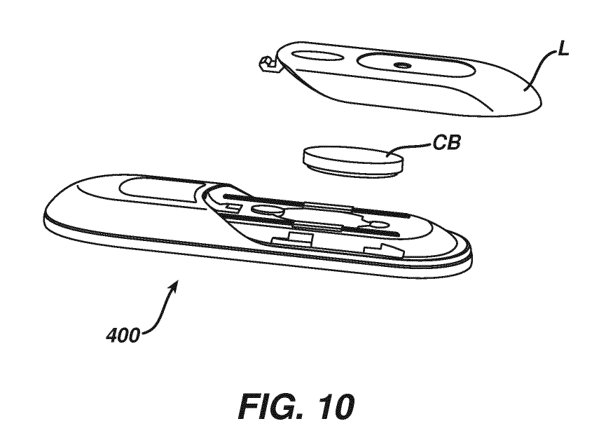

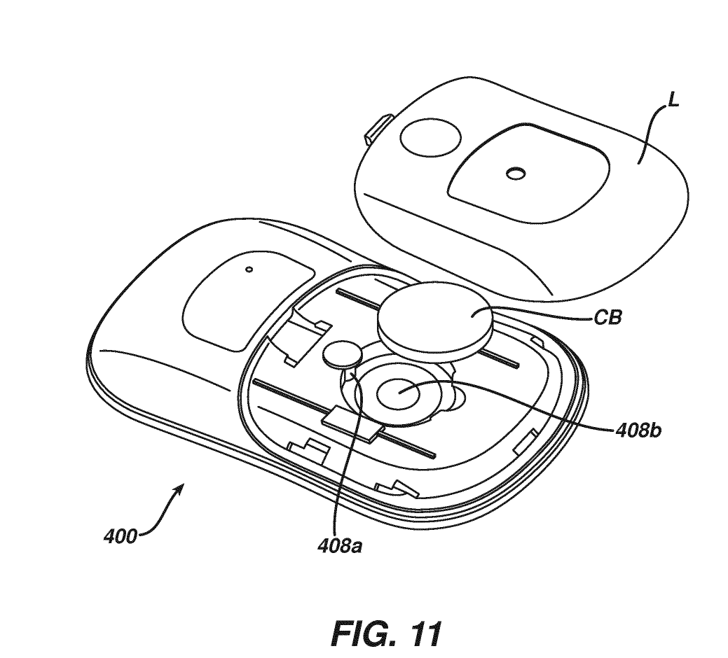

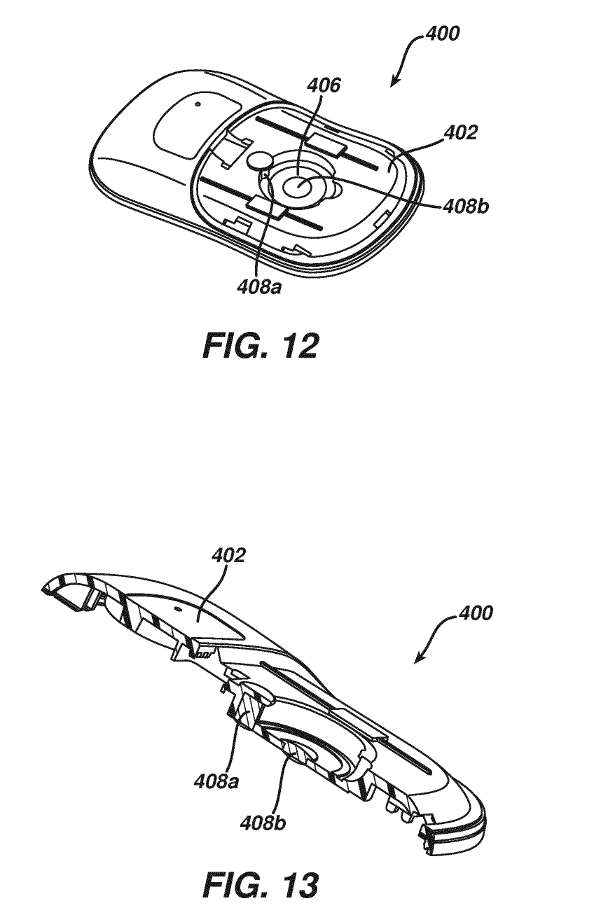

FIG. 10 is a perspective view of a watertight casing with integrated electrical contacts 400 according to a further embodiment of the present invention along with an associated coin cell battery (CB) and lid (L). FIG. 11 is another perspective view of watertight casing 400 along with the associated coin cell battery and lid. Lid (L) is configured to cover the coin cell battery once the coin cell battery has been operably inserted into watertight casing 400 and for simplicity is not depicted in FIGS. 12-21.

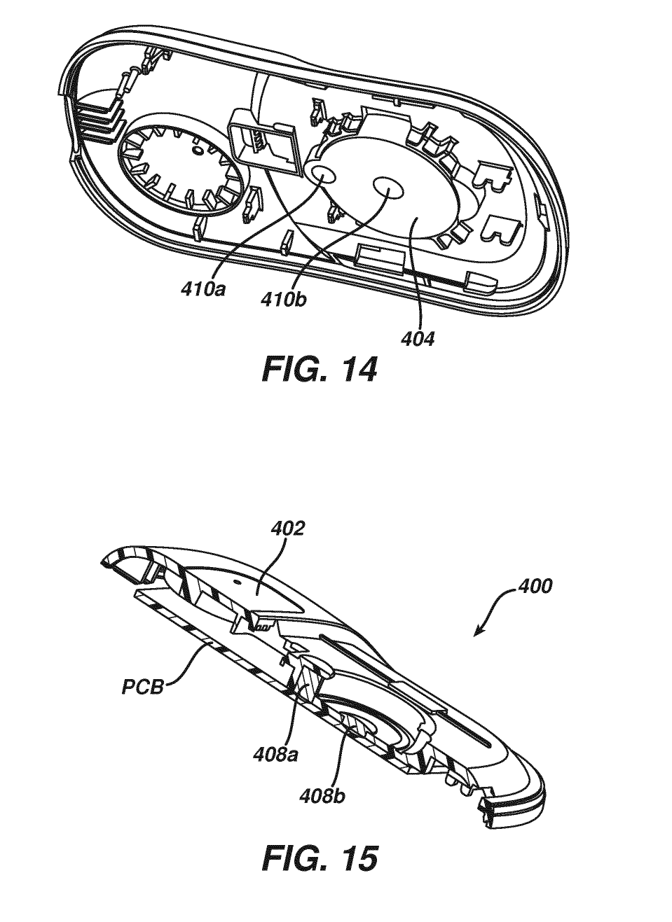

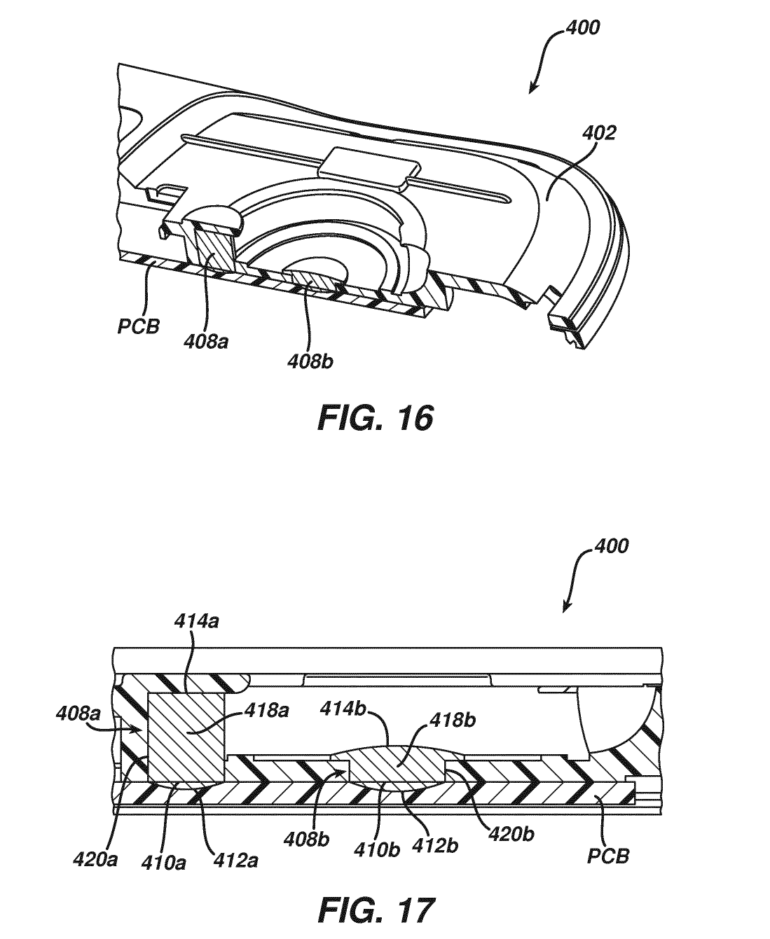

FIG. 12 is a perspective view of watertight casing with integrated electrical contacts 400 (also referred to simply as a “watertight casing”) of FIG. 10. FIG. 13 is a cross-sectional perspective view of watertight casing 400. FIG. 14 is another simplified perspective view of watertight casing 400. FIG. 15 is a cross-sectional perspective view of watertight casing 400 operably contacting a printed circuit board (PCB). FIG. 16 is a cross-sectional perspective view of a portion of watertight casing 400 operably contacting the printed circuit board. FIG. 17 is a cross-sectional view of a portion of watertight casing 400 operably contacting the printed circuit board.

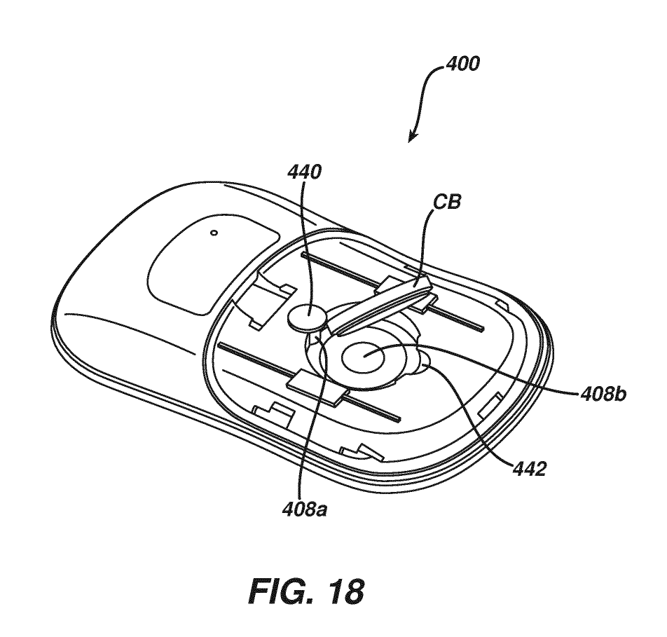

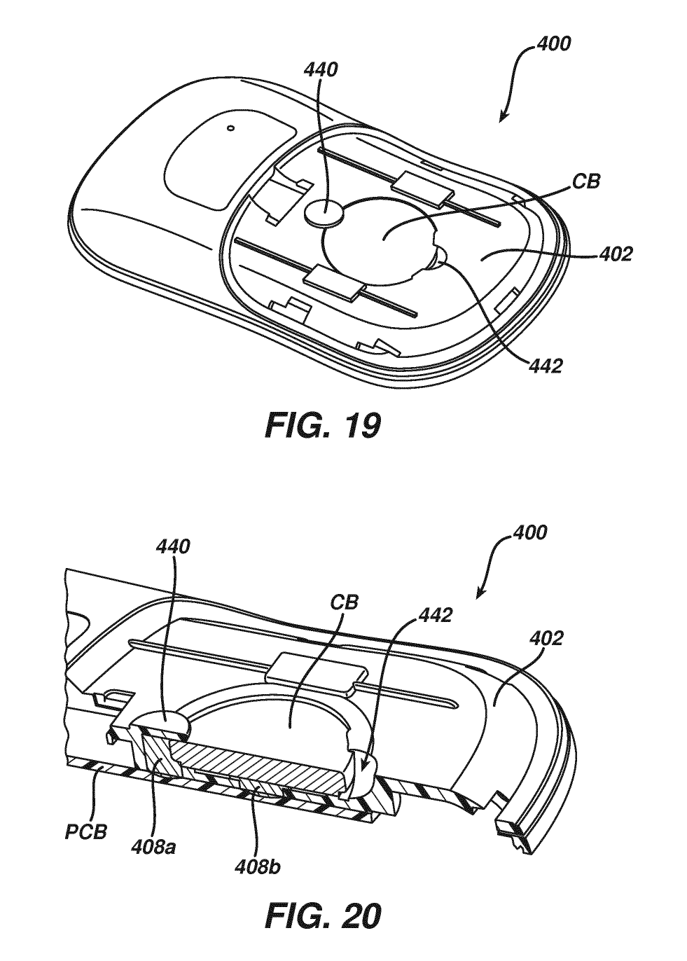

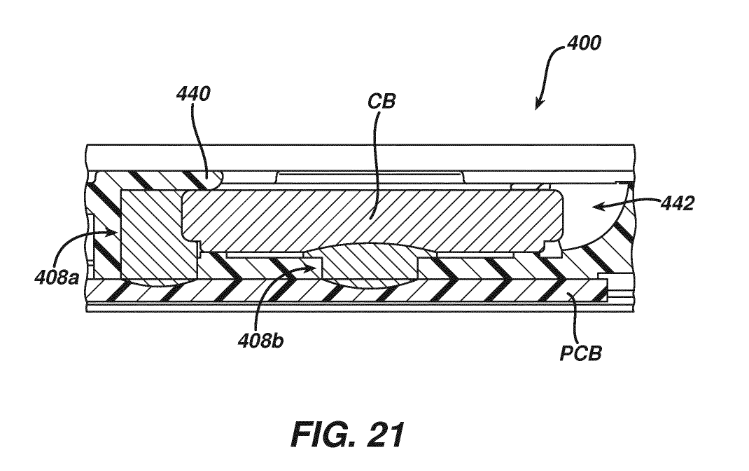

FIG. 18 is a simplified perspective view of watertight casing 400 operably contacting the printed circuit board and with coin cell battery (CB) being inserted therein. FIG. 19 is a simplified perspective view of watertight casing 400 operably contacting the printed circuit board and with the coin cell battery fully inserted therein. FIG. 20 is a simplified cross-sectional perspective view of watertight casing 400 operably contacting the printed circuit board and with the coin cell battery fully inserted therein. FIG. 21 is a simplified cross-sectional view of a portion of watertight casing 400 operably contacting the printed circuit board and with the coin cell battery fully inserted therein.

Referring to FIGS. 10-21, watertight casing with integrated electrical contacts 400 (also referred to simply as “watertight casing”) includes an electrically-insulating plastic case 402 with an inwardly facing surface 404 and an outwardly facing surface 406. Watertight casing 400 also includes two electrically-conductive thermoplastic elastomer (ECTPE) contacts 408a and 408b.

Each of these ECTPE contacts includes a proximal contact portion (410a and 410b, respectively) with a proximal contact surface (412a and 412b, respectively), a distal contact portion (414a and 414b, respectively) with a distal contact surface (418a and 418b, respectively), and a channel portion (420a and 420b, respectively) connecting the proximal contact portion and the distal contact portion of each ECTPE contact (see FIG. 17 in particular). ECTPE contact 408a is configured to connect a negative pole of an inserted battery to a PCB and ECTPE 408b is configured to connect a positive pole of an inserted battery to a PCB (see FIGS. 20 and 21 in particular).

As is evident from FIGS. 10-21, each of the ECTPE contacts is integrated with the electrically-insulating plastic case 402 such that the ECTPE contacts extend through electrically-insulating plastic case 402 from the outwardly facing surface 406 to the inwardly facing surface 404. In addition, each of the proximal contact surfaces (412a and 412b) is disposed in proximity to the inwardly facing surface 404 and the distal contact surfaces (418a and 418b) are disposed in proximity to the outwardly facing surface 406. The distal contact surfaces are disposed such that they make operable electrical contact with the battery that is inserted into watertight casing 400.

Each of the ECTPE contacts (408a and 408b) is adhered to the electrically-insulating plastic case such that a watertight seal between the outwardly facing surface 406 and the inwardly facing surface 404 is present therebetween.

Watertight casing 400 is configured as the upper half of a housing for a hand-held test meter and is of a predetermined geometry for accepting a battery (see FIGS. 18-21 in particular). The configuration (i.e., retention geometry) of the electrically-insulating plastic case is predetermined such that a user must insert the battery as shown by the sequence of images in FIGS. 18-19 and is also intuitively guided to do so. For example, the configuration includes a blocking overhang 440 that prevents incorrect battery insertion that could damage ECTPE contact 408 and a finger slot 442 that enables battery removal by pivoting of the battery away from ECTPE contact 408. These features prevent a user from damaging ECTPE contact 408a by dragging a battery across distal contact surface 418a during insertion and removal.

The elastic nature of the ECTPE contacts is such that the ECTPE contacts elastically deform as the battery is inserted. The ECTPE contacts are of suitable dimensions to apply an appropriate contact force to the battery terminals ensuring good electrical connection.

Simply assembling the hand-held test meter results in the ECTPE contacts making electrical connection with the PCB without the need for soldering. The electrical connection between the ECTPE contacts and the PCB is facilitated by deformation of the ECTPE contacts by, for example, a 50% deformation of the portion of the ECTPE contact that is raised above the inwardly facing surface. Such deformation is not depicted in the simplified FIGs. but would be recognized by one skilled in the art once apprised of the present disclosure. A suitable, but non-limiting, contact resistance between solder pads on the PCB and the ECTPE contacts is approximately 0.1 ohms. However, it is noted that in embodiments of the present invention, the ECTPE contacts need only be deformed by an amount that is adequate to provide an operable contact area, a secure contact, and a suitable contact resistance. The contact and between the ECTPE contacts and a PCB is the result of an applied force that results from the fixing of the watertight casing to the PCB. Such a fixing can be achieved using any suitable mechanical fixing technique such as heat staking or affixing with screws or other fasteners known to one skilled in the art.

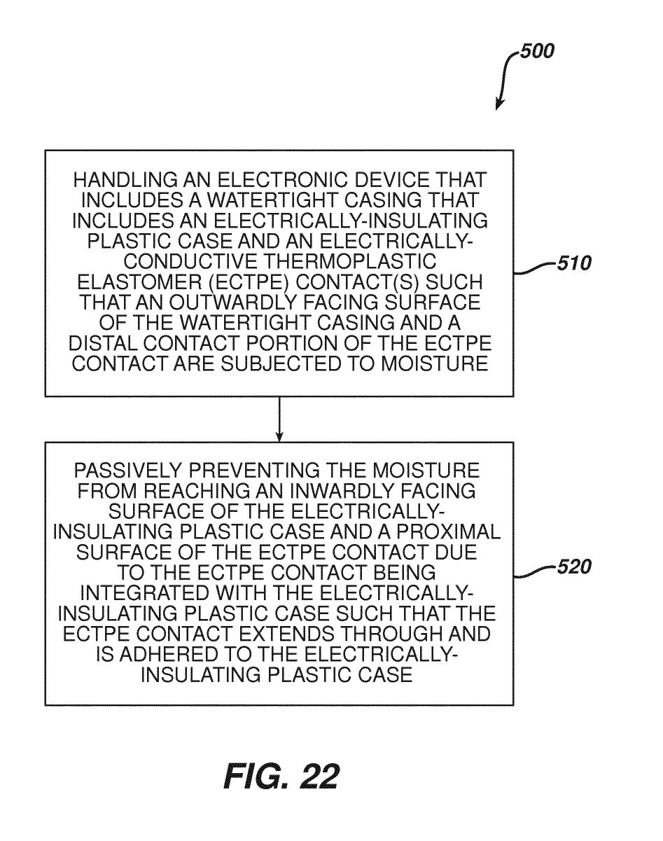

FIG. 22 is a flow diagram depicting stages in a method 500 for handling an electronic device (such as a hand-held test meter for determining an analyte (e.g., glucose) in a bodily fluid (for example, whole blood) sample using an electrochemical-based analytical test strip). Method 500 includes handling an electronic device that includes a watertight casing with an electrically-insulating plastic case and at least one electrically-conductive thermoplastic elastomer (ECTPE) contact, as set forth in step 510.

The handling of step 510 is such that an outwardly facing surface of the watertight casing and a distal contact portion of the ECTPE contact in proximity to the outwardly facing surface are subjected to moisture and/or liquid (for example water, a bodily fluid sample or a suitable cleaning solution). In this regard, an outwardly facing surface that is subjected to liquid is also considered to have been subjected to moisture. Moreover, the term “watertight” as applied to the casing can also include, depending on the degree of adherence between the ECTPE and plastic case, casings that are not only watertight but also airtight.

At step 520 of method 500, the moisture is passively prevented from reaching an inwardly facing surface of the electrically-insulating plastic case and a proximal surface of the ECTPE contact in proximity thereto due to the ECTPE contact being integrated with the electrically-insulating plastic case such that the ECTPE contact extends through the electrically-insulating plastic case from the outwardly facing surface to the inwardly facing surface with the ECTPE contact being adhered to the electrically-insulating plastic case. Moreover, the adherence is such that a watertight seal between the outwardly facing surface and the inwardly facing surface is present therebetween.

If desired, method 500 can be modified to provide for the handling to be such that the outwardly facing surface of the watertight casing and a distal contact portion of the ECTPE contact in proximity to the outwardly facing surface are also subjected to dust, and also so that the preventing is also such that the dust is prevented from reaching an inwardly facing surface of the watertight casing and a proximal surface of the ECTPE contact in proximity thereto in a passive manner due to the ECTPE contact being integrated with the electrically-insulating plastic case.

Once apprised of the present disclosure, one skilled in the art will recognize that method 500 can be readily modified to incorporate any of the techniques, benefits, features and characteristics of water tight casings with integrated electrical contacts according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

1.-21. (canceled)

22. A watertight casing for an electronic device, the watertight casing comprising:
an electrically-insulating plastic case with:
an inwardly facing surface; and
an outwardly facing surface;
at least one electrically-conductive thermoplastic elastomer (ECTPE) contact that includes:
a proximal contact portion with a proximal contact surface;
a distal contact portion with a distal contact surface, and
a channel portion connecting the proximal contact portion and the distal contact portion,
wherein the at least one ECTPE contact is integrated with the electrically-insulating plastic case such that the ECTPE contact extends through the electrically-insulating plastic case from the outwardly facing surface to the inwardly facing surface; and
wherein the proximal contact surface is disposed in proximity to the inwardly facing surface and the distal contact surface is disposed in proximity to the outwardly facing surface; and
wherein the at least one ECTPE contact is adhered to the electrically-insulating plastic case such that a watertight seal between the outwardly facing surface and the inwardly facing surface is present therebetween.

23. The watertight casing of claim 22 wherein the electrically-insulating plastic case is configured as at least a portion of a hand-held test meter for the determination of an analyte in a bodily fluid sample.

24. The watertight casing of claim 23 wherein the electrically-insulating plastic case is configured as a strip port connector of the hand-held test meter.

25. The watertight casing of claim 23 wherein the at least one ECTPE contact is configured to contact an electrochemical-based analytical test strip inserted into the strip port connector and a printed circuit board within the hand-held test meter.

26. The watertight casing of claim 23 wherein the electrically-insulating plastic case is configured as a battery compartment of the hand-held test meter.

27. The watertight casing of claim 26 wherein the plurality of ECTPE contacts are configured to contact a battery inserted into the battery compartment and a printed circuit board within the hand-held test meter.

28. The watertight casing of claim 22 wherein the at least one ECTPE contact is formed of an ECTPE material that includes a non-conductive polymer randomly doped with a conductive particle.

29. The watertight casing of claim 28 wherein the conductive particle is a carbon black particle.

30. The watertight casing of claim 22 wherein the at least one ECTPE material is elastically deformable.

31. The watertight casing of claim 22 wherein the proximal contact portion has a raised profile with respect to the inwardly facing surface and the distal contact portion a raised profile with respect to the outwardly facing surface.

32. The watertight casing of claim 31 wherein at least the proximal contact portion and the distal contact portion are elastically deformable.

33. A method for handling an electronic device, the method comprising:
handling an electronic device that includes a watertight casing with:
an electrically-insulating plastic case; and
at least one electrically-conductive thermoplastic elastomer (ECTPE) contact,
such that an outwardly facing surface of the watertight casing and a distal contact portion of the ECTPE contact in proximity to the outwardly facing surface are subjected to moisture, and
preventing the moisture from reaching an inwardly facing surface of the electrically-insulating plastic case and a proximal surface of the ECTPE contact in proximity thereto in a passive manner due to the ECTPE contact being integrated with the electrically-insulating plastic case such that the ECTPE contact extends through the electrically-insulating plastic case from the outwardly facing surface to the inwardly facing surface with the ECTPE contact being adhered to the electrically-insulating plastic case such that a watertight seal between the outwardly facing surface and the inwardly facing surface is present therebetween.

34. The method of claim 33 further wherein the handling is also such that the outwardly facing surface of the watertight casing and a distal contact portion of the ECTPE contact in proximity to the outwardly facing surface are subjected to dust, and
the preventing is also such that the dust is prevented from reaching an inwardly facing surface of the watertight casing and a proximal surface of the ECTPE contact in proximity thereto in a passive manner due to the ECTPE contact being integrated with the electrically-insulating plastic case.

35. The method of claim 33 wherein the electrically-insulating plastic case is configured as at least a portion of a hand-held test meter for the determination of an analyte in a bodily fluid sample.

36. The method of claim 33 wherein the electrically-insulating plastic case is configured as a strip port connector of the hand-held test meter.

37. The method of claim 36 wherein the at least one ECTPE contact is configured to contact an electrochemical-based analytical test strip inserted into the strip port connector and a printed circuit board within the hand-held test meter.

38. The method of claim 33 wherein the electrically-insulating plastic case is configured as a battery compartment of the hand-held test meter.

39. The method of claim 38 wherein the plurality of ECTPE contacts are configured to contact a battery inserted into the battery compartment and a printed circuit board within the hand-held test meter.

40. The method of claim 33 wherein the at least one ECTPE contact is formed of an ECTPE material that includes a non-conductive polymer randomly doped with a conductive particle.

41. The method of claim 40 wherein the conductive particle is a carbon black particle.

42. The method of claim 33 wherein the at least one ECTPE material is elastically deformable.
